(19) United States
(12) Patent Application Publication
(10) Pub. No.: US 2021/0234702 A1
(43) Pub. Date: Jul. 29, 2021

(54) MULTI-DECENTRALIZED PRIVATE BLOCKCHAINS NETWORK

(71) Applicant: OMNIVEK AG, Luzern (CH)

(72) Inventor: Sergey BEKIYANTS, Holmdel, NJ (US)

(21) Appl. No.: 17/051,044

(22) PCT Filed: Apr. 29, 2019
(86) PCT No.: PCT/US19/29726

§ 371 Date: Oct. 27, 2020

Related U.S. Application Data
(60) Provisional application No. 62/663,894, filed on Apr. 27, 2018.

(51) Int. Cl. H04L 9/32; G06F 21/60; G06F 16/27; G06F 16/23; H04L 29/08

(52) U.S. Cl. CPC H04L 9/3239; G06F 21/602; H04L 2209/38; G06F 16/2379; H04L 67/10; G06F 16/27

(57) ABSTRACT

A decentralized private blockchains network is provided. In some implementations, the system performs operations comprising receiving, at a client system, data having a sensitivity level, the operations further comprise splitting, at the client system, the data based on the sensitivity level, the splitting comprising splitting the data into data portions. The operations further comprise storing, at a decentralized storage system, the decentralized storage system comprising a plurality of private blockchains, each of the data portions in a separate private blockchain of the plurality of private blockchains. The operations further comprise storing, at a multi-decentralized private blockchains network (MDPBN), pointers to locations of the data portions within the decentralized storage system. Related systems, methods, and articles of manufacture are also described.

### CROSS-REFERENCE TO RELATED APPLICATIONS

This application is a national stage application, filed under 35 U.S.C. § 371, of International Patent Application No. PCT/US2019/029726 filed on Apr. 29, 2019, and claims the benefit of priority to U.S. Provisional Patent Application Ser. No. 62/663,894, filed on Apr. 29, 2018, the contents of which are hereby incorporated by reference herein in their entirety.

### TECHNICAL FIELD

The present disclosure relates to the field of blockchain technology and, more specifically, to secure private distributed ledger technology.

### BACKGROUND

A blockchain is a growing list of records or data blocks, which are linked together using cryptography to form a blockchain. Each block may contain a cryptographic hash of the previous block, a timestamp, and transaction data. A blockchain is typically managed by a peer-to-peer computing network in which the nodes in the network collectively adhere to a protocol for inter-node communication and validating new blocks. Once recorded, the data in any given block cannot be altered retroactively without alteration of all subsequent blocks and the consensus of the network majority. As such, a blockchain may be used to produce an immutable record or ledger for the data recorded in each block in the blockchain.

One application of blockchain technology is to safeguard and allow for the creation and distribution of digital currency. While the popularity and value of digital currencies have risen over the course of the last number of years, their usability and convenience has not. The number of people who own digital assets has increased substantially, but those people are unable to realize the full value and usability of those digital currencies in the real world. Purchasing goods and services with these assets is extremely difficult even in the rare cases where businesses do accept them.

By extension, the difficulty in the use of digital currency, restricts e-commerce activity. For example, business owners who want to accept payments using digital currencies, do not have access to any consistent and viable methods or procedures to convert digital currency to fiat currency. There is a need for highly secure computing infrastructures, in which digital confirmations and transactions can be efficiently managed and completed.

### SUMMARY

In accordance with one or more implementations, a multi-decentralized private blockchains network (MDPBN) is provided for utilizing multi-secure technology that supports multiple currencies (digital and fiat), using dynamic biometric verification and instant multi-signature transaction confirmations on a Hyperledger/InterPlanetary File System (IPFS) Platform. This implementation allows users to store their digital assets with unmatched security and execute instant currency conversions and confirmations at rates of more than 100,000 transactions per second. The proprietary platform is also able to provide secure know your customer (KYC) and key recovery service (KRS) modules that are stored on their own blockchain network within the MDPBN.

An MDPBN may be created as a transactional data storage, retrieval, and confirmation system to support various aspects of currency use (e.g., buying, selling, trading, and exchanging digital and fiat currency on a B2C, B2B, and e-commerce platform) in the financial technology market, including, for example, the creation of a digital currency exchange, as well as the storage and certification of sensitive information (e.g., KYC, KRS, identification documents, personal or corporate legal documents, biometric data, and various forms of information storage).

In certain embodiments, multiple forms of biometric data such as voice samples, facial features, eye maps, fingerprints, dynamic facial movement, and DNA fractions may be stored in the MDPBN. An individual's biometric data, for example, may be uploaded to the blockchain network and split into redundant fragments stored in separate parts of the multi-layered blockchains network along with portions offline, controlled by data splitters. A confirmation (e.g., a “notary function”) may be generated by the system when certain data is present or stored on the multilayer blockchain. When requested, the data comprising the confirmation may be retuned in a sequence according to the provisions of a smart contract. The data may be reconstructed for use to validate access or confirm transactions. Original data elements may not be used to validate access or confirm a transaction. Depending on implementation, the confirmation may be the single element needed for validation or confirmation. In this fashion, the original data elements need not be reconstructed in the system, and instead the confirmation of their presence would be used to ensure the security of the blockchain system.

In some aspects, a method, computer program product and system are provided. In an implementation, a MDPBN system is provided. The system can include (or otherwise utilize) at least one processor and/or memory, which can be configured to perform operations including receiving, at a client system, data having a sensitivity level. The operations may further include splitting the data based on the sensitivity level at the client system. The splitting may comprise splitting the data into data portions. The operations may further include storing, at a decentralized storage system, a plurality of private blockchains. At least one of the data portions may be stored in a separate private blockchain of the plurality of private blockchains. The operations may further include storing, at a MDPBN, pointers to locations of the data portions within the decentralized storage system.

In some variations, the data may include, for example, passport data, license information, or other types of legal or non-legal information. In some variations, the data stored in the decentralized data storage comprises personal data, keys to digital wallets, know your customer data, key recovery details, documents, biometric data, and audio or video files. In some variations, the biometric data may be selected from fingerprints, DNA information for humans or animals, voice data, and dynamic facial movement data, for example.

In certain embodiments, the client computer system may further comprise a data builder configured to retrieve, from the multi-decentralized private block chains network, the pointers to the data portions. The data builder may be further configured to request, from the decentralized storage system and based on the pointers, the data portion action stored within the separate private block chains. The data builder may be further configured to receive, from the decentralized storage system in response to the request, the data portions. The data builder may be further configured to construct the data from the received data portions.

In some variations, the MDPBN comprises multiple peer levels or layers. In one embodiment, a first level of peers may include encrypted pointers to the data portions stored in the decentralized storage system, a second level of peers may store data and validate transactions on the first level network, and a third level of peers may participate in the storage of encrypted data. The participating may comprise syncing data between multiple peers within a blockchain, using the second level of peers to retrieve conditions, validating the transaction, and writing the result of the validation to the decentralized storage system.

In some variations, the first level of peers is configured to define conditions to retrieve data in the MDPBN. In some variations, the first level of peers shares at least one peer with the decentralized storage system. In some variations, the decentralized storage system includes the second level of peers and the third level of peers.

Depending on implementation, the approach and methodology used to build MDPBN applications may comprise performing individual application functionality in a separate independent decentralized private blockchains network. Transactional or sensitive data may be split-up and stored between several private blockchains, which may be accessible if there is a condition (e.g., “a trigger”) in another blockchain.

Based on an identifiable condition or trigger, the split-up data can be reconstituted from multiple blockchain networks. Depending on implementation, different conditions or rules may be utilized or programmed to retrieve data from multiple private blockchains, such as a specific transaction in a ledger, multi-signature smart contracts requiring (n of m keys), multiple blockchains network conditions, as well as any user-defined conditions in a smart contract. In certain embodiments, redundant data elements may be stored in the multiple blockchains (e.g., online or offline) to provide a more reliable data retention platform by preventing data loss that may be caused by hardware malfunction or loss of connectivity.

### BRIEF DESCRIPTION OF THE DRAWINGS

The accompanying drawings, which are incorporated in and constitute a part of this specification, show certain aspects of the subject matter disclosed herein and, together with the description, help explain some of the principles associated with the disclosed implementations as provided below.

FIG. 1 is a block diagram of an example peer-sharing platform, in accordance with one or more embodiments.

FIG. 2 is a block diagram of a sensitive storage cluster for managing the storage and retrieval of sensitive data in a distributed data storage environment implemented over a blockchain network, in accordance with one or more embodiments.

FIG. 3 is a block diagram of a main blockchain network configured for storing portions of sensitive data to a sensitive storage cluster, in accordance with one or more embodiments.

FIG. 4 is a block diagram of a data vault system in accordance with certain aspects of the present disclosure.

FIG. 5 is a diagram of the components of the data vault system including a data splitter in accordance with certain aspects of the present disclosure.

FIG. 6 is a diagram illustrating a method of exchanging cryptographic information in a MDPBN, in accordance with certain aspects of the present disclosure.

FIG. 7 is a diagram of create file communication exchange in a data vault system in accordance with certain aspects of the present disclosure.

FIG. 8 is a diagram of a read file communication exchange in a data vault service in accordance with certain aspects of the present disclosure.

FIG. 9 is a diagram of an update file communication exchange in the data vault service in accordance with certain aspects of the present disclosure.

The figures may not be to scale in absolute or comparative terms and are intended to be exemplary. The relative placement of features and elements may have been modified for the purpose of illustrative clarity. Where practical, the same or similar reference numbers denote the same or similar or equivalent structures, features, aspects, or elements, in accordance with one or more embodiments.

### DETAILED DESCRIPTION

In the following, numerous specific details are set forth to provide a thorough description of various embodiments. Certain embodiments may be practiced without these specific details or with some variations in detail. In some instances, certain features are described in less detail so as not to obscure other aspects. The level of detail associated with each of the elements or features should not be construed to qualify the novelty or importance of one feature over the others.

In accordance to one or more embodiments, the disclosed subject matter may be implemented using one or more public or private blockchain networks. In one implementation, one or more decentralized peer-to-peer networks may be utilized in which one or more participants maintain a replica of a shared ledger of digitally signed transactions. The replicas may be synchronized through a protocol referred to as consensus. Certain guarantees on the immutability of the ledger may be provided, even if some participants in the application of the consensus protocol are malicious.

Depending on implementation, there may be restrictions on the type and number of participants in the network, the execution of the consensus protocols, or the manner in which the shared ledger is maintained. For example, in an embodiment in which a public blockchain is utilized, participation may be open to anyone. In contrast, in a private blockchain, the participants may be limited to a number of parties that agree to a defined consensus protocol. Advantageously, a private blockchain may be configured to be faster, more secure, and better suited for enterprise use than a public counterpart.

Systems and methods disclosed herein may utilize a private multi-layer blockchain, for example, by partitioning transaction information across multiple blockchains, using a Data Splitter (DS) and a Data Builder (DB), for example. The DS may be used to split transaction data and distribute the corresponding data bytes (e.g., along with an imbedded trigger condition) with a unique hash in multiple storage blockchain networks. Different trigger conditions combined with a smart contract may be configured to retrieve data from the multiple blockchains using a DB, as provided in further detail herein.

Because storing sensitive or private information in a centralized third-party repository may create significant risks of loss or breach, in accordance with example embodiments, one or more private keys (e.g., secret passphrases, strings of random characters, or grouping of random words) may be utilized to limit access to sensitive or private information or digital assets. Certain embodiments may include a key recovery service (KRS), which enables users to recover their assets in the event their private keys are lost or stolen. Users may thus reclaim assets contained within a digital wallet, even in cases of catastrophe, such as when private keys are lost.

To provide a robust mechanism for recovery, a private key may be split in multiple portions. The multiple portions may be duplicated for the purpose of redundancy and be kept securely in, for example, one or more offline and independently maintained storage areas or machines. Such independent storage implementation may ensure that the private keys, or portions thereof, are securely stored as independent redundant fragments, such that if one fragment is lost a corresponding duplicate redundant fragment can be used instead.

When first setting up a digital wallet, users may be requested to enter personal information or answer security questions. The provided information and answers may be stored in the MDPBN to form the basis for a KYC service. In addition or instead, users may set up dynamic biometric data set, also stored in the MDPBN. The biometric data may be used to verify the user and sign or confirm the transaction. Together, these security hurdles form the basis for a multi-secure blockchain network. For added security, the private keys may be generated using a computing system that is functionally and communicatively independent (e.g., disconnected from any computing network or other computing system). Should a user wish to recover a lost private key, the user may use a multi-factor verification or authentication system that would provide the user with the lost key after authenticating the user based on the user's biometrics, for example.

The biometrics may be based on a user's personal information (e.g., fingerprint, facial features, DNA, voice, etc.) that can verify the user's identity to the MDPBN's KYC service. This verification and authentication may be the entry point for other services provided by the MDPBN. The personal data may be stored in a secure blockchain network to protect the personal data from loss or misuse. After personal data is verified, the KYC service can validate the user's identity to one or more MDPBN services or platforms without disclosing any of the user's personal data to other blockchain channels. Accordingly, once a user's biometrics are verified by the KYC, the user may have access to a variety of MDPBN services.

Biometrics-based verification may utilize a group of private blockchain channels in the MDPBN. A private blockchain channel may be responsible for a specific type of biometric data (e.g., dynamic facial movement, voice, fingerprint, etc.). A user's biometric template may be encrypted and stored using biometric encryption, untraceable, and cancelable biometrics, which may involve an intentional and systematically repeatable distortion of biometric features and digital key features in order to protect sensitive user-specific data. Additional data (e.g., helper data) may be utilized to securely bind a digital key to a biometric, or extract a key from the biometric, so that neither the key nor the biometric can be retrieved from a stored biometric encryption template. Biometric encryption may display a false rejection rate (FRR) or a false acceptance rate (FAR), which may be improved by the use of MDPBN's multi-factor verification.

In accordance with one or more embodiments, to address security and scalability problems in a digital currency exchange, instead of implementing a centralized exchange infrastructure, a MDPBN may be implemented as a transactional data storage, data retrieval, and confirmation system. A MDPBN may be for example implemented as a blockchain-based, decentralized data storage system capable of storing unlimited amounts of data including personal data, keys to digital wallets, know your customer data, key recovery details, sensitive documents including passport, licenses, legal documents, biometric data including fingerprints, DNA information for humans or animals, voice data, dynamic facial movement data, and ocular biometrics, and audio or video files.

The systems and methods described herein may also include a wallet provider service (WPS) that generates and stores multiple user keys. Users may submit requests to the WPS for transactions using digital currency while simultaneously generating a key on the user's device. Once the user has signed the transaction with his private key, the WPS signs the transaction and pushes the transaction to external blockchains for verification. These actions, including the transaction hash initiated by the WPS, may be saved on the MDPBN to ensure confirmation, verification, and key security. In example scenarios where a user has lost their device and has to make a multifactor verification request through KYC and KRS to restore digital assets, WPS uses its key alongside with the key stored within the KRS to sign a recovery transaction. This request and all associated information may be saved on the MDPBN in order to prove the usage of the WPS key.

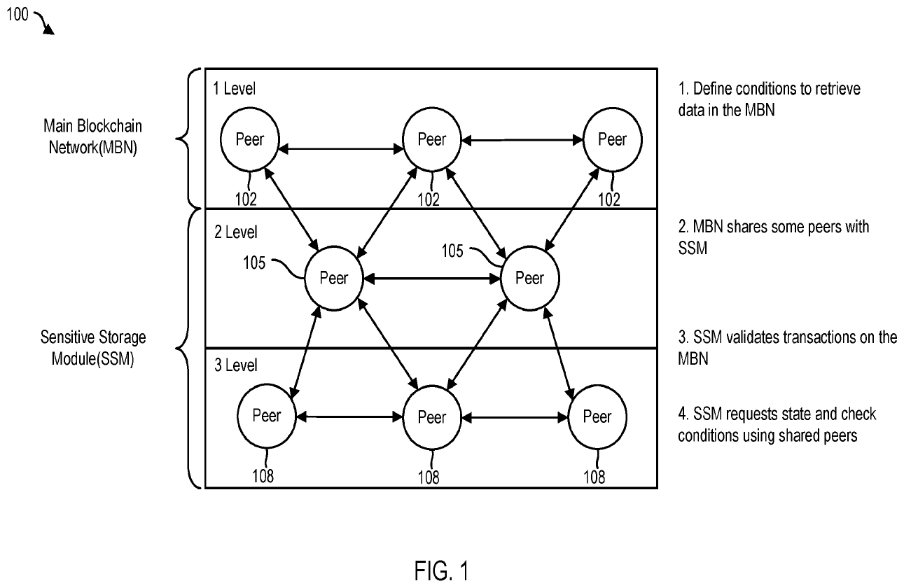

FIG. 1 is an example block diagram of a peer-to-peer data sharing platform, in accordance with one or more embodiments, comprising a main blockchain network (MBN) and a sensitive storage module (SSM) in a multi-level peer network implemented for peer sharing, transaction confirmation, and ledger synchronization. As shown, the peer-sharing platform may include three levels of peers, for example. A first level of peers 102 contains encrypted pointers or references to portions of data in one or more SSMs. A second level of peers 105 may store data and validate transactions over the first level peers. A third level peers 108 may participate in the storage of encrypted data.

Data stored in the third level peers 108 may be split into multiple portions by way of a DS (not shown in FIG. 1). The DS may split data into smaller data portions based on one or more factors. One factor may be the sensitivity or importance of the data. For example, highly sensitive data such as personally identifiable information (PID (e.g., a social security number, a passport number, etc.) may be split into one or more smaller data portions. Splitting data into multiple smaller portions allows the smaller portions of data to be stored in a distributed manner. The more the data is split, the smaller is the chance for data theft or data misappropriation because it would be more difficult for an unscrupulous attacker to piece together the various portions of the data stored in a distributed storage environment.

In some aspects, one or more blockchains in the MDPBN may add a unique hash value to the split data portions. The data portions, or pointers to the data portions, may be stored in the SSM to indicate the location of various portions of the original data split into multiple portions. The multiple data portions or the pointers may be utilized to reconstruct the original data, when the original data is to be retrieved. In some implementations, the SSM may comprise a database or repository on a client device or server. The SSM may be implemented as an independent unit in a sensitive storage cluster having a first series of peers, unique cryptographic mechanism, and API. An SSM may encapsulate sets of data portions, or pointers to the sets of data portions, without knowledge of data portions stored in others SSMs.

In accordance with one or more embodiments, third level peers 108 may sync data between multiple peers 108 within a blockchain, may use the second level peers 105 to retrieve conditions, validate the transaction, and write the result of the validation to the SSM. In some aspects, the blockchain network storing sensitive data may retrieve sensitive data portions from storage and reconstitute the data portions into the original sensitive data based on the occurrence of a triggering condition being met, for example.

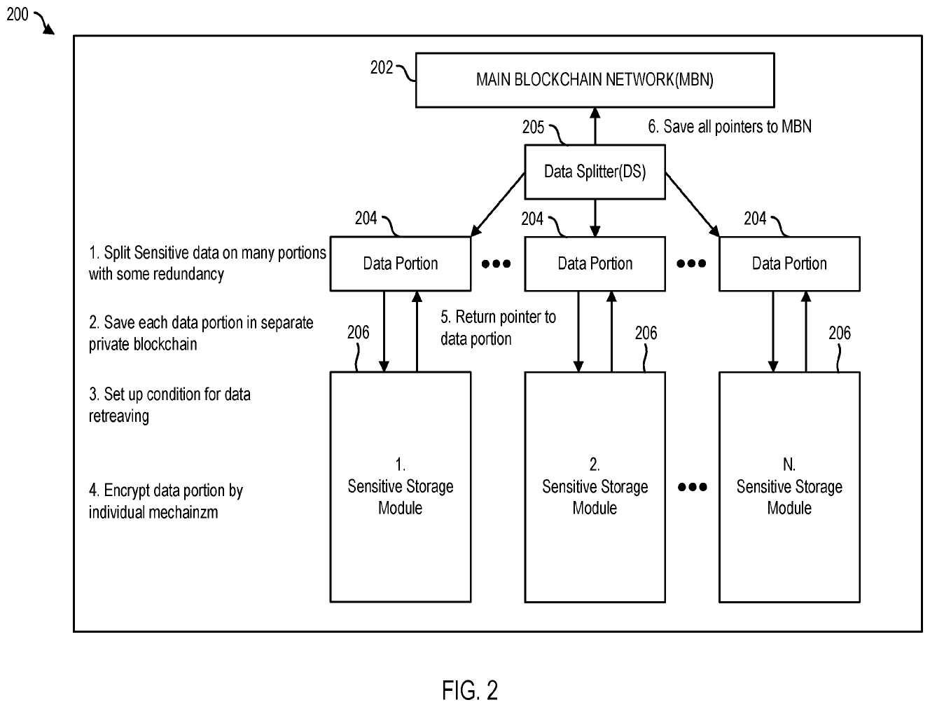

FIG. 2 illustrates a block diagram of at least one sensitive storage cluster (SSC) 200, including a cluster of SSMs comprising a plurality of modules. As shown, the SSC 200 includes a DS 205 configured to split data (e.g., based on a sensitivity level) to be stored and may leverage the split data portions' distribution through multiple SSMs in the SSC 200. The DS 205 may split data into data bytes (e.g., data portions 204). A blockchain may add unique hash values to the data portions 204, and the data portions 204 may be stored in SSMs 206. DS 205 may save pointers or references to the different data portions 204 within the MBN 202. The SSMs 206 may be constructed on a private blockchain separate or independent from MBN 202.

The SSM 206 may, for example, be implemented as an independent unit in the SSC 200 with its own peers, unique cryptographic mechanism, and API. An SSM 206 may encapsulate sets of data portions 204 without knowledge of data portions stored in others SSMs 206. As data portions 204 are stored into the different SSMs 206, the SSMs 206 may return pointers to the data portions 204 back to the DS 205. The pointers to the data portions 204 may be stored in the MBN 202. The pointers may include references or links to the locations of the data portions 204 within the respective SSM 206. Additionally, the DS 205 may configure conditions for retrieving the data portions 204. In some aspects, the conditions may include requests from a user for personal information or requests from a smart contract to verify a signature or a party to the contract. Because different portions of sensitive data are stored across different block chains with different encryptions, the SSC 200 provides increased security for sensitive information.

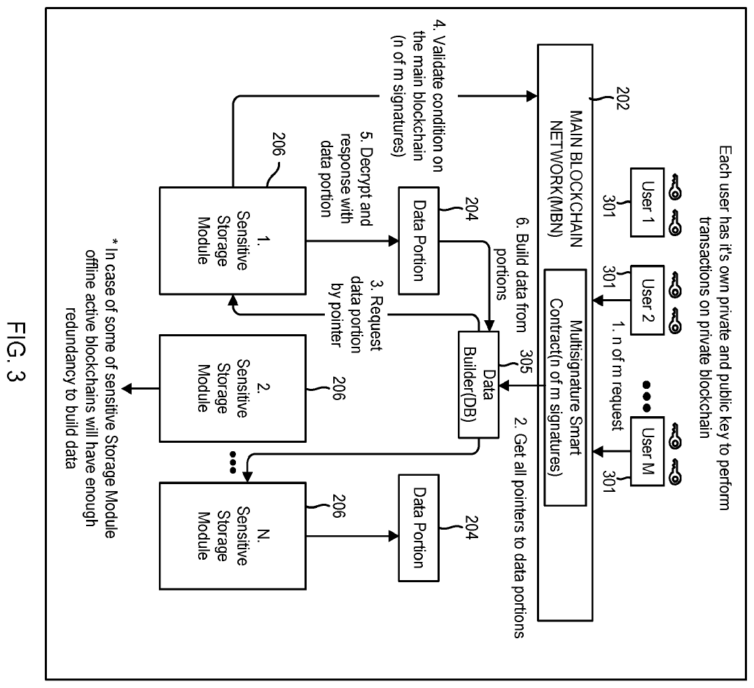

FIG. 3 illustrates a diagram of a multi-signature smart contract (MBN) 300 in communication with users 301 of the MBN 202, which may be a private blockchain that trusts storing portions of sensitive data to the SSC containing SSMs 206. The MBN 202 stores encrypted pointers to data portions 204. As shown, the MBN 202 includes a multi-signature smart contract 303. In some aspects, conditions to reconstruct or retrieve data previously split by the data splitter 205 and stored and encrypted by the SSMs 206 may be managed by the smart contract 303. Multi-signature requests of the smart contract 303 may be one of the conditions to retrieve data.

In one embodiment, DB 305 may retrieve the pointers to the data portions needed by the smart contract 303 from the MBN 202. DB 305 may transmit a request to SSMs 206 in the SSC 200 to retrieve the various portions of the requested data stored in a distributed manner in multiple SSMs 206. Such requests may include pointers to the data portions. When the SSM 206 receives the request, SSM 206 may decrypt the request with the pointer, retrieve the data portion based on the pointer, and return the data portion to DB 305. Once the data portions for a requested data are retrieved, DS 305 may combine the data portions to reconstruct the requested data. Introducing data redundancy in the SSC 200 (with certain logic) may allow for DB 305 to reconstruct the requested data even when one or more of the SSMs 206 may be unavailable, corrupted or otherwise compromised. SSM 206 peers may participate in transaction validation within their own blockchain network and at the MBN 202, and syncs the ledger on both chains.

In accordance with example an implementation, a multi-secure technology including N levels of security may be provided to protect users and their digital assets. In an example embodiment in which N=5, five levels of security may be implemented and the various levels may include an MDPBN (e.g., MBN 202), multi-signature transactions (e.g., smart contract 303), dynamic biometrics-based verification (e.g., KRS/KYC), peer-to-peer blockchain roles and permissions such as a peer-sharing process 100 and multi-factor authorization (e.g., KRS/KYC). When combined, the N levels of security form the basis of the MDPBN's multi-secure platform which makes the platform very secure.

The embodiments described herein may be applied in a variety of implementations. For example and with reference to FIGS. 1 through 3, a user 301 of the MDPBN may be entering sensitive personally identifiable information (PID at a client system.

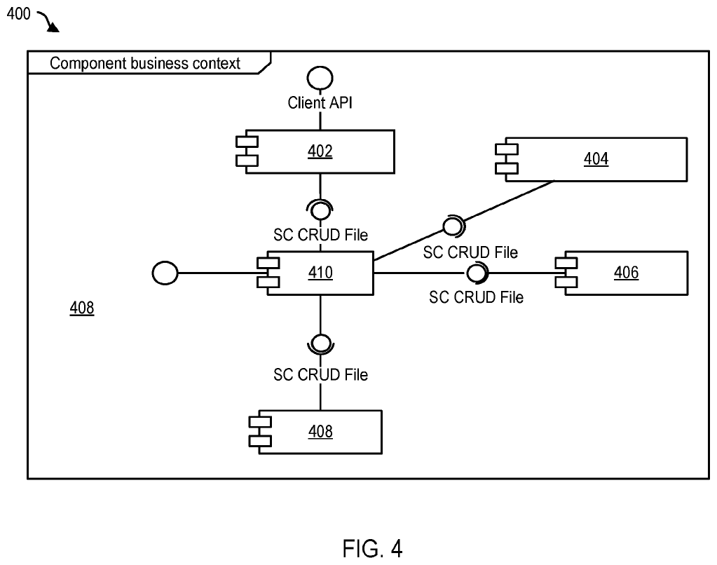

FIG. 4 illustrates a MultiDecentralized Data Vault (MDDV) environment 400. As shown in FIG. 4, the MDDV 400 comprises a client organization 402, a monitoring organization 404, a first data processor 406, a data vault 410, and a second data processor 408. The client organization 402 may include a client application programming interface (API). As shown, the client organization 402 may upload data (e.g., a SC CRUD file) to the data vault 410. The SC CRUD file may comprise a smart contract Create Read Update Delete (CRUD) file. After uploading the SC CRUD file, the monitoring organization 404 may perform transaction validation on distributed ledger networks for the benefit of the client organization 402. As shown in FIG. 4, the monitoring organization 404 retrieves the SC CRUD file to perform validation. Additionally, the first data processor 406 may retrieve the SC CRUD file to perform transaction validation on distributed ledger networks. The second data processor 408 may retrieve the SC CRUD file and may also perform transaction validation on distributed ledger networks.

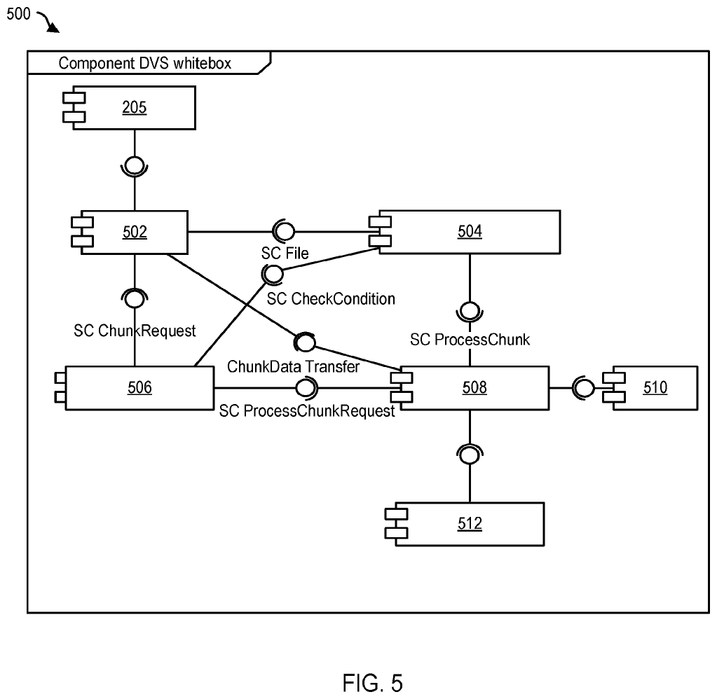

FIG. 5 illustrates a Data Vault service (DVS) 500. The DVS 500 comprises the data splitter 205, a multi-decentralized data vault software development kit (MDDV SDK) 502, a multi-decentralized data vault distributed ledger (MDDV DL) network 504, a data vault service distributed ledger (DVS DL) network 506, a DVS backend 508, and HSM 510, and a long-term data storage 512. With reference to FIG. 2, the data splitter 205 may split data into data portions 204 with a chosen level of redundancy and may recombine an array of data portions 204 to form the original data based on certain conditions being met. The MDDV SDK 502 may include a client-side software development kit to communicate within the DVS 500. The MDDV DL network 504 may comprise DVS instances configured to store an original data chunk (e.g., data portion 204) and provide permission-based access to it. The DVS backend 508 may comprise a non-deterministic logic of a data vault service to receive, encrypt, save chunk data and retrieve, decrypt and send it to a requester on a successful ChunkRequest. He HSM 510 may comprise a hardware security module configured to manage digital keys which are used to encrypt and decrypt chunk data. The long-term data storage 512 may comprise Key-Value storage for encrypted chunk data. As shown in FIG. 5, the smart contract file is exchanged between MDDV SDK 502 and the MDDV DL network 504. MDDV SDK 502 also transfers chunk data (ChunkDataTransfer) to the DVS backend 508. Additionally, the MDDV SDK 502 transmits a chunk request (SC ChunkRequest) to the DVS DL network 506. The MDDV DL network 504 sends a smart contract read request (SC ProcessChunk) to the DVS backend 508. The MDDV DL network 504 also executes an SC CheckCondition to verify that a file recondition is met and sends the results to the DVS DL network 506. The DVS backend 508 then transmits some key-value data to the long-term data storage 512 and digital key data to the hardware security module (HSM) 510.

In one aspect, the user may be entering a passport number to upload to the MDPBN. Upon uploading the passport number, the DS 205 at the client system may split the data into smaller data portions 204 based on a determined sensitivity level of the passport number. In some implementations, the user 301 may select the sensitivity level of the passport number or other information entered into the system. In one implementation, the higher the sensitivity level, the more data portions the DS 205 will split the data into. In some aspects, have a large number of data portions 204 may make it harder for a hacker to determine the original data because the hacker will have to overcome the encryptions of each data portion 204 stored in the SSC 200 and respective SSMs 206. Pointers to the location of each data portion 204 may be stored in the MBN 202.

In one or more embodiments, the DS 205 may also configure trigger conditions to retrieve the split data portions 204. For example, if a user loses a private key or the private key is stolen, the user may be requested to enter personal information, or answers to security questions, or biometric data, including voice and dynamic biometrics, for example, in order to recover the private key and access to digital assets. When the trigger conditions have been satisfied, the data builder 305 may retrieve the pointers to the data portions 205 from the MBN 202. The data builder 305 may then send requests to each SSM 206 in the SSC 200 that this is storing the data portions 204 of the original data (e.g., passport number).

The data builder 305 may determine the appropriate SSM 206 to contact based on the pointers retrieved from the MBN 202. Upon receipt of the request from the data builder 305, the SSM 206 may decrypt the request to retrieve a corresponding data portion 204 and return the data portion 204 to the data builder 305. The data builder 305 may combine the data portions 204 received to reconstruct the original data. For example, the data splitter may split the passport number into individual digits and store each digit of the passport number in a different SSM 206. After responding to the request from the data builder 305, the SSM 206 may transmit the digits back to the DB 305 in the data builder 305, which may combine the separate digits of the passport number to determine the original passport number.

Accordingly, the retrieval of sensitive information of the user 301 can be accomplished utilizing the MDPBN, without having to expose any of the user's personal data to any other blockchain channel. When the user needs to provide proof of requested data, the user may confirm the requested data to the service or transaction requiring it through dynamic biometrics-based verification.

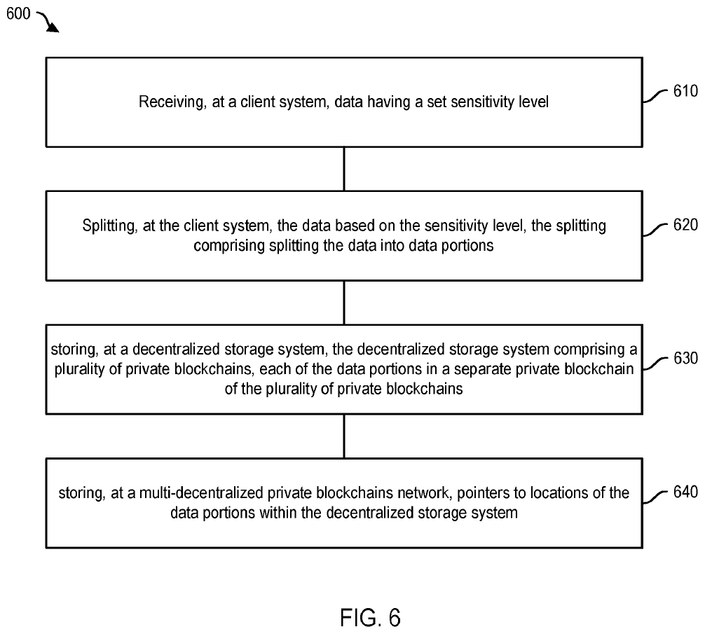

FIG. 6 is a diagram illustrating a method 600 of exchanging cryptographic information in a MDPBN, in accordance with certain aspects. One or more of the steps described in the method 600 of FIG. 6 may be performed by a client system 402, the data splitter 205, the DVS 500, a server, or any other similar device. At block 610, the method includes receiving, at a client system, data having a sensitivity level. At block 620, the method includes splitting, at the client system, the data based on the sensitivity level, the splitting comprising splitting the data into data portions. At block 630, the method includes storing, at a decentralized storage system, the data portions, such that the data portions are stored in a separate private blockchain of the plurality of private blockchains, where the decentralized storage system comprising a plurality of private blockchains. At block 640, the method includes storing, at a MBN, pointers to locations of the data portions within the decentralized storage system.

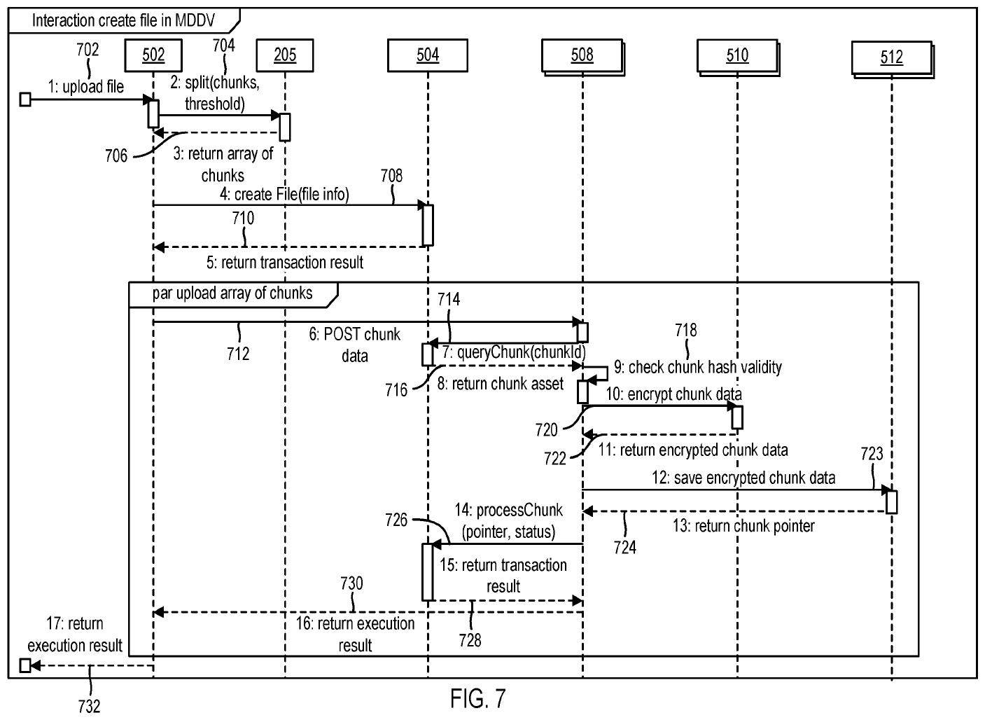

FIG. 7. Is a diagram of create file communication exchange 700 in the data vault service (e.g., DVS 500) in accordance with certain aspects of the present disclosure. In some aspects, the communication exchange 700 includes executing a create file in the MDDV (e.g. MDDV 400). At 702, a client system (e.g. client organization 402) uploads a file to the MDDV SDK 502. At 704, the data splitter 205 splits the original data (e.g. the uploaded file) into data chunks or data portions 204 based on a sensitivity level. At 706, the data splitter 205 returns an array of data chunks or data portions 204 back to the MDDV SDK 502. At 708, the MDDV SDK 502 creates a file and transmits the file (e.g., SC File) to the MDDV DL network 504. The MDDV DL network 504, at 710, returns the transaction result to the the MDDV SDK 502. At 712, the MDDV SDK 502 post chunk data to the DVS backend 508. The DVS backend 508, at 714, queries for a chunk (queryChunk) to the MDDV DL network 504. At 716, the MDDV DL network 504 returns the chunk asset to the DVS backend 508. At 718, the DVS backend 508 checks the chunk hash validity. At 720, the DVS backend 508 encrypts the chunk data and transmits it to the HSM 510. At 722, the HSM 510 returns the encrypted chunk data to the DVS backend 508. At 723, the DVS backend 508 saves the encrypted chunk data to the long-term data storage 512. At 724, the long-term data storage 512 returns a pointer to the chunk data to the DVS backend 508. At 726, the DVS backend 508 sends a ProcessChunk interface to the MDDV DL network 504 which includes the pointer to the data chunk and a status. At 728, the MDDV DL network 504 returns a transaction result to the DVS backend 508. At 730, the DVS backend 508 returns the execution result to the MDDV SDK 502. At 732, the MDDV SDK 502 returns the execution result to the client system.

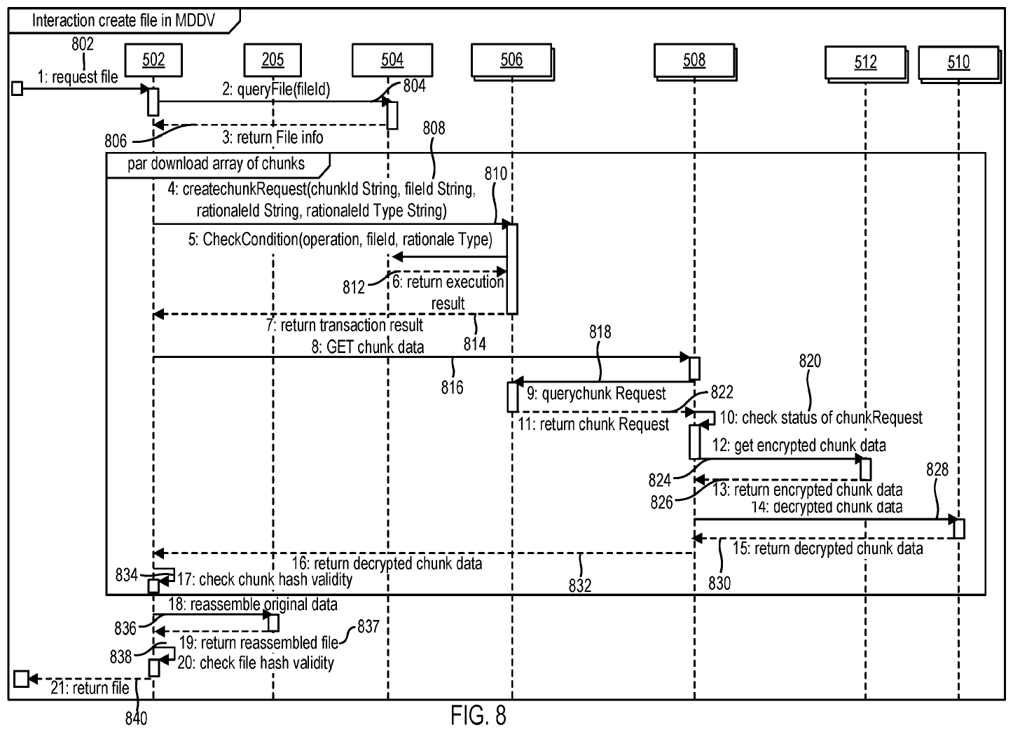

FIG. 8 is a diagram of a read file communication exchange 800 in the data vault service (e.g., DVS 500) in accordance with certain aspects of the present disclosure. As shown in FIG. 8, at 802, a client system (e.g., client organization 402) may request the file from the MDDV SDK 502. At 804, the MDDV SDK 502 queries a file from the MDDV DL network 504. At 806, the MDDV DL network 504 returns the file information back to the MDDV SDK 502. At 808, the MDDV SDK 502 creates a chunk requests for chunk data from the DVS DL network 506. At 810, the DVS DL network 506 sends a check condition interface to the MDDV DL network 504. At 812, the MDDV DL network 504 returns execution result for the check condition to the DVS DL network 506. At 814, the DVS DL network 506 returns a transaction result to the MDDV SDK 502. At 816, the MDDV SDK 502 transmits a get chunk data request to the DVS backend 508. At 818, the DVS backend 508 transmit a queryChunkRequest to the DVS DL network 506. At 820, the DVS backend 508 checks the status of the chunk request. At 822, the DVS backend 508 returns the chunk request to the DVS DL network 506. At 824, the DVS backend 508 sends a get encrypted chunk data request to the long-term data storage 512. At 826, the long-term data storage 512 returns the encrypted chunk data back to the DVS backend 508. At 828, the DVS backend 508 sends decrypted chunk data to the HSM 510. At 830, the HSM 510 returns the decrypted chunk data back to the DVS backend 508. At 832, DVS backend 508 returns the encrypted chunk data to the MDDV SDK 502. At 834, the MDDV SDK 502 checks the chunk data hash for validity. At 836, the MDDV SDK 502 sends a request to reassemble the original data to the data splitter 205. At 837, the data splitter 205 returns the assembled file to the MDDV SDK 502. At 838, the MDDV SDK 502 checks the file hash validity of the assembled file. At 840, the MDDV SDK 502 returns the file back to the client system.

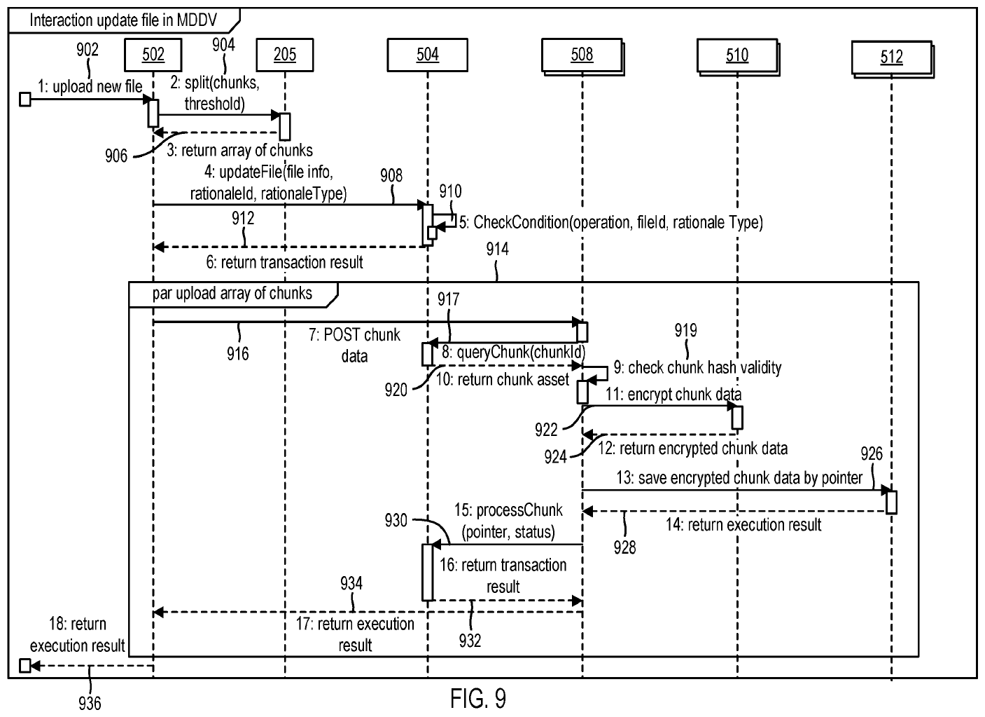

FIG. 9 is a diagram of an update file communication exchange 900 in the data vault service (e.g., DVS 500) in accordance with certain aspects of the present disclosure. As shown in FIG. 9, at 902, a client system may request a file update from the MDDV SDK 502. At 904, the MDDV SDK 502 sends the file to the data splitter 205 to split the data into data chunks, or data portions 204. At 906, the data splitter 205 returns an array of data chunks or data portions 204 back to the MDDV SDK 502. At 908, the MDDV SDK 502 sends an update file request to the MDDV DL network 504. At 910, the MDDV DL network 504 performs a check condition operation on the file. At 912, the MDDV DL network 504 returns the transaction result back to the MDDV SDK 502. At 914, the MDDV SDK 502 sends a post chunk data command to the DVS backend 508. At 918, the DVS backend 508 sends a queryChunk command to the MDDV DL network 504. At 919, the DVS backend 508 checks the chunk hash validity. At 920, the MDDV DL network 504 returns the chunk asset to the DVS backend 508. At 922, the DVS backend 508 sends a command to encrypt chunk data to the HSM 510. At 924, the HSM 510 returns the encrypted chunk data to the DVS backend 508. At 926, DVS backend 508 sends a command to save encrypted chunk data by pointer to the Long term data storage 512. At 928, the long-term data storage 512 returns execution result activity to the DVS backend 508. At 930, the DVS backend 508 sends a process chunk request to the MDDV DL network 504. At 932, the MDDV DL network 504 returns the transaction result to the DVS backend 508. At 934, the DVS backend 508 returns the execution result to the MDDV SDK 502. At 936, the MDDV SDK 502 returns the execution result to the client system.

### Terminology

When a feature or element is herein referred to as being “on” another feature or element, it may be directly on the other feature or element or intervening features and/or elements may also be present. In contrast, when a feature or element is referred to as being “directly on” another feature or element, there may be no intervening features or elements present. It will also be understood that, when a feature or element is referred to as being “connected”, “attached” or “coupled” to another feature or element, it may be directly connected, attached or coupled to the other feature or element or intervening features or elements may be present. In contrast, when a feature or element is referred to as being “directly connected”, “directly attached” or “directly coupled” to another feature or element, there may be no intervening features or elements present.

Although described or shown with respect to one embodiment, the features and elements so described or shown may apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments and implementations only and is not intended to be limiting. For example, as used herein, the singular forms “a”, “an” and “the” may be intended to include the plural forms as well, unless the context clearly indicates otherwise. It will be further understood that the terms “comprises” and/or “comprising,” when used in this specification, specify the presence of stated features, steps, operations, processes, functions, elements, and/or components, but do not preclude the presence or addition of one or more other features, steps, operations, processes, functions, elements, components, and/or groups thereof. As used herein, the term “and/or” includes any and all combinations of one or more of the associated listed items and may be abbreviated as “/”.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” Use of the term “based on,” above and in the claims is intended to mean, “based at least in part on,” such that an unrecited feature or element is also permissible.

Spatially relative terms, such as “forward”, “rearward”, “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features due to the inverted state. Thus, the term “under” may encompass both an orientation of over and under, depending on the point of reference or orientation. The device may be otherwise oriented (rotated 90 degrees or at other orientations) and the spatially relative descriptors used herein interpreted accordingly. Similarly, the terms “upwardly”, “downwardly”, “vertical”, “horizontal” and the like may be used herein for the purpose of explanation only unless specifically indicated otherwise.

Although the terms “first” and “second” may be used herein to describe various features/elements (including steps or processes), these features/elements should not be limited by these terms as an indication of the order of the features/elements or whether one is primary or more important than the other, unless the context indicates otherwise. These terms may be used to distinguish one feature/element from another feature/element. Thus, a first feature/element discussed could be termed a second feature/element, and similarly, a second feature/element discussed below could be termed a first feature/element without departing from the teachings provided herein.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise.

For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, may represent endpoints or starting points, and ranges for any combination of the data points. For example, if a particular data point “10” and a particular data point “15” may be disclosed, it is understood that greater than, greater than or equal to, less than, less than or equal to, and equal to 10 and 15 may be considered disclosed as well as between 10 and 15. It is also understood that each unit between two particular units may be also disclosed. For example, if 10 and 15 may be disclosed, then 11, 12, 13, and 14 may be also disclosed.

Although various illustrative embodiments have been disclosed, any of a number of changes may be made to various embodiments without departing from the teachings herein. For example, the order in which various described method steps are performed may be changed or reconfigured in different or alternative embodiments, and in other embodiments, one or more method steps may be skipped altogether. Optional or desirable features of various device and system embodiments may be included in some embodiments and not in others. Therefore, the foregoing description is provided primarily for the purpose of example and should not be interpreted to limit the scope of the claims and specific embodiments or particular details or features disclosed.

The examples and illustrations included herein show, by way of illustration and not of limitation, specific embodiments in which the disclosed subject matter may be practiced. As mentioned, other embodiments may be utilized and derived therefrom, such that structural and logical substitutions and changes may be made without departing from the scope of this disclosure. Such embodiments of the disclosed subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve an intended, practical or disclosed purpose, whether explicitly stated or implied, may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

The disclosed subject matter has been provided here with reference to one or more features or embodiments. Those skilled in the art will recognize and appreciate that, despite of the detailed nature of the example embodiments provided here, changes and modifications may be applied to said embodiments without limiting or departing from the generally intended scope. These and various other adaptations and combinations of the embodiments provided here are within the scope of the disclosed subject matter as defined by the disclosed elements and features and their full set of equivalents.

What is claimed is:

1. A system for providing a cryptographic platform for exchanging information, the system comprising:
a client computer system, the client computer system comprising a data splitter configured to split, based on a sensitivity level of data, data received at the client computer system into smaller data portions;
a multi-decentralized private blockchains network (MDPBN) configured to store pointers to the data portions;
a decentralized storage system comprising a plurality of private blockchains, the decentralized storage system configured to store the data portions in separate private blockchains, each of the separate private block chains having different encryption mechanisms.

2. The system of claim 1, wherein the data comprises passport, licenses, and/or legal information.

3. The system of claim 1, wherein the data stored in the decentralized data storage comprises at least one of personal data, keys to digital wallets, know your customer data, key recovery details, document, biometric data, and audio or video files.

4. The system of claim 1, wherein the biometric data is selected from fingerprints, DNA information for humans or animals, voice data, and dynamic facial movement data.

5. The system of claim 1, the client computer system further comprising:
a data builder configured to:
retrieve, from the multi-decentralized private block chains network, the pointers to the data portions;
request, from the decentralized storage system and based on the pointers, the data portion action stored within the separate private block chains;
receive, from the decentralized storage system in response to the request, the data portions; and
construct the data from the received data portions.

6. The system of claim 1, wherein the MDPBN comprises at least three levels of peers, wherein the first level of peers include encrypted pointers to the data portions stored in the decentralized storage system, the second level of peers store data and validate transactions on the first level network, and a third level of peers that participate in the storage of encrypted data, and wherein the participating comprises syncing data between multiple peers within a blockchain, using the second level of peers to retrieve conditions, validating the transaction, and writing the result of the validation to the decentralized storage system.

7. The system of claim 6, wherein the first level of peers are configured to define conditions to retrieve data in the MDPBN.

8. The system of claim 6, wherein the first level of peers share at least one peer with the decentralized storage system.

9. The system of claim 6, wherein the decentralized storage system includes the second level of peers and the third level of peers.

10. A method of exchanging cryptographic information, the method comprising:
receiving, at a client system, data having a sensitivity level;
splitting, at the client system, the data based on the sensitivity level, the splitting comprising splitting the data into data portions;
storing, at a decentralized storage system, the decentralized storage system comprising a plurality of private blockchains, each of the data portions in a separate private blockchain of the plurality of private blockchains; and
storing, at a multi-decentralized private blockchains network (MDPBN), pointers to locations of the data portions within the decentralized storage system.

11. The method of claim 10, further comprising:
receiving, at the MDPBN, a request for the data;
retrieving, at a data builder of the client system, the pointers to the locations of the data portions;
requesting, by the data builder and based on the pointers, the data portions stored within the decentralized storage system; and
receiving, at the data builder, the data portions from the decentralized storage system; and
combining, by the data builder, the data portions to construct the data.

12. The method of claim 10, wherein the data stored in the decentralized data storage comprises personal data, keys to digital wallets, know your customer data, key recovery details, document, biometric data, and audio or video files.

13. The method of claim 10, wherein the biometric data is selected from fingerprints, DNA information for humans or animals, voice data, and dynamic facial movement data.

14. The method of claim 10, wherein the MDPBN comprises at least three levels of peers, wherein the first level of peers include encrypted pointers to the data portions stored in the decentralized storage system, the second level of peers store data and validate transactions on the first level network, and a third level of peers that participate in the storage of encrypted data, and wherein the participating comprises syncing data between multiple peers within a blockchain, using the second level of peers to retrieve conditions, validating the transaction, and writing the result of the validation to the decentralized storage system.

15. The method of claim 14, wherein the first level of peers are configured to define conditions to retrieve data in the MDPBN.

16. The method of claim 14, wherein the first level of peers share at least one peer with the decentralized storage system.

17. The method of claim 10, wherein the sensitivity level comprises a user entered sensitivity level.

18. The method of claim 10, wherein storing each of the data portions comprises storing redundant data portions in separate parts of the decentralized storage system along with storing redundant data portions offline.

19. A non-transitory computer program product storing instructions which, when executed by at least one data processor, causes operations comprising:
receiving, at a client system, data having a sensitivity level;
splitting, at the client system, the data based on the sensitivity level, the splitting comprising splitting the data into data portions;
storing, at a decentralized storage system, the decentralized storage system comprising a plurality of private blockchains, each of the data portions in a separate private blockchain of the plurality of private blockchains; and
storing, at a multi-decentralized private blockchains network (MDPBN), pointers to locations of the data portions within the decentralized storage system.

20. The computer program product of claim 19, wherein the operations further comprise:
receiving, at the MDPBN, a request for the data;
retrieving, at a data builder of the client system, the pointers to the locations of the data portions;
requesting, by the data builder and based on the pointers, the data portions stored within the decentralized storage system; and
receiving, at the data builder, the data portions from the decentralized storage system; and
combining, by the data builder, the data portions to construct the data.
